(19) United States
(12) Patent Application Publication
(10) Pub. No.: US 2007/0149883 A1
(43) Pub. Date: Jun. 28, 2007

(54) Method for detecting heart beat and determining heart and respiration rate

(76) Inventor: Itshak Ben Yesha, Moshav Shilat (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 10/596,996

(22) PCT Filed: Feb. 9, 2005
(86) PCT No.: PCT/IL05/00158

§ 371 Date: Jul. 5, 2006

(30) Foreign Application Priority Data
Feb. 10, 2004 (IL) 160308

(51) Int. Cl. A61B 5/02

(52) U.S. Cl. 600/485

(57) ABSTRACT

Disclosed is an apparatus and system for non-invasively detecting and determining the heart rate and respiration rate of a patient, while the patient is within their sleep environment, suitable for both home and hospital monitoring, which includes an array of at least two pressure-sensitive sensors, positioned under the mattress, which gathers data from the patient corresponding to the vertical and horizontal movements of the body, and wherein the data from each sensor is collected, filtered, and analyzed and finally, the difference between the results gathered from each sensor detects and determines heart and respiration rates.

### FIELD OF INVENTION

This invention relates generally to vital sign detectors, and specifically to devices used to non-invasively detect the heart and respiration rates of patients in a bed or other sleep environment.

### BACKGROUND OF THE INVENTION

There are many patents that monitor a patient's vital signs. Such prior art heart rate detection monitors are often invasive, requiring that the patient make physical contact with the sensors.

The heart rate is the number of contractions of the heart in one minute and it is measured in beats per minute (bpm). When resting, the adult human heart beats at about 70 bpm (males) and 75 bpm (females), but this rate varies between individuals.

The body can increase the heart rate in response to a wide variety of conditions in order to increase the cardiac output (the amount of blood ejected by the heart per unit time). Exercise causes a normal person's heart rate to increase above the resting heart rate. As the physical activity becomes more vigorous, the heart rate increases. With very vigorous exercise, a maximum heart rate can be reached.

The pulse is the most straightforward way of measuring the heart rate. The pulse rate can be measured at any point on the body where an artery is close to the surface, including the wrist (radial artery), neck (carotid artery), elbow (brachial artery), and groin (femoral artery).

Another method of measuring heart rate is using commercially available heart rate monitors, which employ a chest strap to sense the heart rate using electrodes to acquire the electrical activity of the heart, and a wrist receiver for displaying the signal. These monitors allow accurate measurements to be continuously taken and can be used during exercise when manual measurement would be difficult or impossible (such as when the hands are being used).

In hospitals, an electrocardiograph is frequently used to measure and monitor heart rates by applying electrodes to the patient's chest, in order to ascertain the electrical activity of the heart.

The circulatory system or cardiovascular system is the organ system that circulates blood through the human body. Oxygenated blood from the lungs returns to the heart via the pulmonary veins, flows into the left atrium and then into the left ventricle, which then pumps the blood through the aorta, the major artery that supplies blood to the body.

“Blood flow” is the flow of blood in the cardiovascular system wherein:

$$F = \frac{Δ P}{R}$$

$$and$$

$$R = ( \frac{vL}{{r}^{4}} ) ( \frac{8}{π} )$$

where F is the blood flow, P is the pressure and R is the resistance. The blood flow depends on the pressure difference in the vascular system. The flow of the blood through human body during circulation can be described as the movement of fluid mass across, and mainly along, the body.

There are several patents related to the measurements of heartbeat and respiration. U.S. Pat. No. 5,448,996 relates to a patient monitor sheet device of simplified construction which permits the accurate measurement of respiration, heart beat, and body position with a minimum of intrusion on the subject. Sensors are located in a bed sheet with which a subject comes in contact. One sensor produces a signal corresponding to respiratory induced, pulmonary motion, and myocardial pumping sounds. A second sensor produces a signal corresponding to changes in body position. A processor amplifies and filters the induced signals resulting in resolved output highly correlated to respiration rate, heart beat rate, and changes in body position. This device requires that the patient be in contact with the sensors and additionally relies on boosting signals to provide the required information.

U.S. Pat. No. 4,738,264 discloses a device for sensing heart and breathing rates in a single transducer and having electronic and filtering circuits to process the electrical signal generated by the transducer. The transducer is an electromagnetic sensor constructed to enhance sensitivity in the vertical direction of vibration produced on a conventional bed by the action of patient's heartbeat and breathing functions and achieves sufficient sensitivity with no physical coupling between the patient resting in bed and the sensor placed on the bed away from the patient. The electronic circuits integrate the electrical energy generated by the sensor that pertains to cardiac and breathing information and sets off an alarm when pre-set circuits of these functions have been surpassed. The device has applications in monitoring SIDS (Sudden Infant Death Syndrome) and non-ambulatory patients. But this device must combine collected data to detect heartbeat and breathing rates

U.S. Pat. No. 6,278,890 provides a non-invasive methodology and instrumentation for the detection and localization of abnormal blood flow in a vessel of a patient. An array of sensors is positioned on an area of a patient's body above a volume in which blood flow may be abnormal. Signals detected by the sensor array are processed to display an image that may indicate the presence or absence of abnormal blood flow. However, there is no ability to detect and determine heart rate.

U.S. Pat. No. 5,479,932 provides an apparatus for monitoring the health of an infant, realized by simultaneously detecting large motor movement, heart beat and respiration of the infant, and sounding an alarm when an exacting combination of all three signals is not sensed. This integrated combination eliminates false alarms inherent in prior art monitors. Preferably, a passive sensor is placed under, but not in direct contact with, a child for generating a voltage in proportion to the movement of the child. This signal is amplified, filtered, and analyzed for the presence of large motor movement, heartbeat, and respiration.

U.S. Pat. No. 6,547,743 is a movement sensitive mattress that has a plurality of independent like movement sensors for measuring movement at different locations on the mattress to generate a plurality of independent movement signals. The signals are processed to derive respiratory variables including rate, phase, maximum effort, or heart rate. Such variables can be combined to derive one or more diagnostic variables including apnea and labored breathing classifications. This device is able to determine heart rate; however, it depends on combined data to determine that rate.

### SUMMARY OF INVENTION

The present invention demonstrates a non-invasive method and system for using blood mass circulation to detect the presence of a heartbeat and determine the heart rate. The same method and system applies to using the movement of the diaphragm to detect respiration and determine respiration rate.

Blood enters the right side of the heart through two veins: the superior vena cava (SVC) and the inferior vena cava (IVC). The SVC collects blood from the upper half of the body. The IVC collects blood from the lower half of the body. Blood leaves the SVC and the IVC and enters the right atrium (RA). When the RA contracts, the blood goes through the tricuspid valve and into the right ventricle (RV). When the RV contracts, blood is pumped through the pulmonary valve, into the pulmonary artery (PA) and into the lungs where it picks up oxygen. Blood now returns to the heart from the lungs by way of the pulmonary veins and goes into the left atrium (LA). When the LA contracts, blood travels through the mitral valve and into the left ventricle (LV). The LV is a very important chamber that pumps blood through the aortic valve and into the aorta. The aorta is the main artery of the body and receives all of the blood that the heart has pumped out and distributes it to the rest of the body. The LV has a thicker muscle than any other heart chamber because it must pump blood to the rest of the body against much higher pressure in the general circulation (blood pressure).

The present invention discloses a unique monitor, with a new method and system for detecting the presence of a heartbeat and determining the heart and respiration rate of a patient while the patient is in a sleep environment. This method is based on comparing the pressure changes induced by blood mass circulation through the patient's body by at least two pressure-sensitive sensors located under the mattress of the patient. The differences of the detected signals between individual sensors or groups of sensors monitor heartbeat and diaphragm movements and provide heart and respiration rates.

The heart and respiration rates are determined by a subtraction of the pressure signals corresponding to the upper body and the lower body of the patient and mathematically determining the maximum difference of signal between each group of sensors. The accuracy of this device makes it suitable for use in hospital monitoring, while remaining simple to operate and inexpensive, making it also suitable for home use.

There is the option of using this device with existing respiratory monitoring technology. It is possible to use either the sum of the signals, or their difference, in order to detect the presence of respiration. The sum of the signals corresponds to the vertical movements of the chest, and the difference of the signals corresponds to the axial movements of the diaphragm.

It is possible to use the same or additional sensors for an optional presence detection system that shuts off the alarm system if the patient is not on the bed, in order to assist in preventing false alarms. The same presence detection system will enable an “absence” alarm when the invention is embedded in a remote monitoring system.

### BRIEF DESCRIPTION OF DRAWINGS

These and further features and advantages of the invention will become more clearly understood in light of the ensuing description of a preferred embodiment thereof, given by way of example only, with reference to the accompanying drawings, wherein—

FIG. 1: shows a perspective view of the sensors and pad.

FIG. 2: is a side view of the present invention, in place under a mattress.

FIG. 3A: shows a graph of signals collected by a prior art systems over a half-second period.

FIG. 3B: shows a graph of signals collected a by prior art systems over a ten-second period.

FIG. 3C: shows a graph of data collected by the present invention over the same ten-second period.

### DESCRIPTION OF THE PREFERRED EMBODIMENT

Disclosed is an apparatus and system with a novel, non-invasive method for detecting the heartbeat and determining the heart rate of a patient within their sleep environment. The apparatus also detects the presence of respiration and is able to determine respiration rate.

Basic to the system of the present invention is an array of at least two pressure-sensitive sensors (3), located under the patient's body, which gather data from the patient, corresponding to the movements of the patient's body, including blood movement caused by circulation. The data from each sensor (3) is collected and undergoes the process of digitizing. Next, the difference between the results gathered from each sensor-group is filtered and analyzed. The analyzed difference determines the presence or absence of a heartbeat, as well as the actual heart and respiration rates. This system is suitable for both home and hospital monitoring, and can function as a cardio-respiratory monitor, analyzing the signals with a dedicated set of filters and functions. The results may be displayed on the device itself or can be transmitted to other equipment.

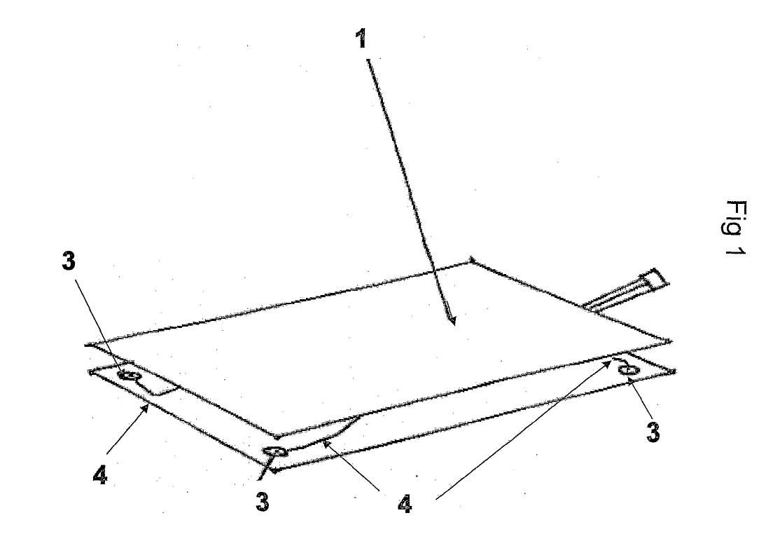

Referring now to FIG. 1, at least two pressure sensitive, piezoelectric sensors (3) are inserted within a protective pad (1). These pressure sensitive sensors (3) use existing technology to gather signals generated by vertical and horizontal movements from the body of the patient, especially blood circulation, chest movements, and diaphragm movements, and by vertical environmental noises.

The pad (1) consists of two solid boards, located under the mattress of the patient, between which are placed an array of pressure sensitive sensors (3). The pad (1) covers the part of the bed under the space occupied by the patient's body with the option of manufacturing the apparatus to fit any size needed to match the patient's size and sleep environment.

The sensors (3) are situated in the pad (1) such that one sensor (3) monitors body movements and changes of pressure caused by blood circulation across the lower part of the patient's body and the second sensor (3) monitors body movements and changes of pressure caused by blood circulation across the upper part of the patient's body. There is also the option of using three or more sensors (3). In an alternative embodiment, two pairs of sensors (3) are placed in the pad (1) as described above. In yet another embodiment, sensors (3) are placed in various locations in the pad (1), ensuring that the patient is continuously monitored, even when the patient changes position on the bed.

Each sensor (3) is individually connected to a cable (4). The cables (4), in turn, are connected to a processing and control unit (2). The cables (4) transmit the data collected by the sensors (3) and reflect those changes in pressure to the processing and control unit (2). The processing and control unit (2) analyzes all of the gathered data in order to detect the patient's heartbeat and then determine the heart rate. The control unit simultaneously utilizes the same data to detect the presence of respiration and determine the respiration rate.

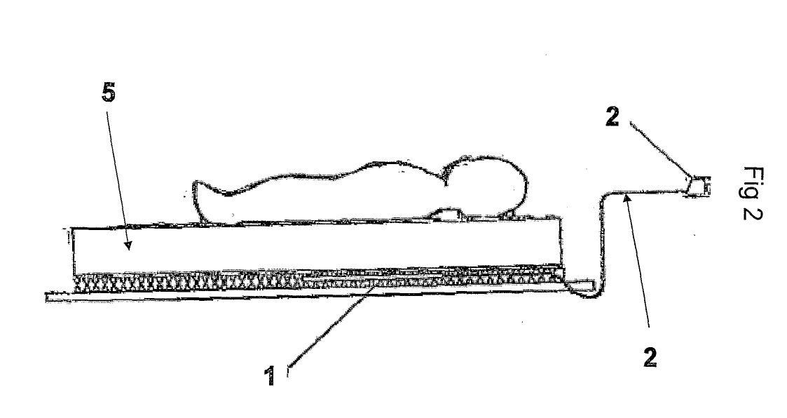

FIG. 2 shows the placement of all of the components of the apparatus on a patient bed. The pad (1), with the sensors (3) between the two boards, is placed on a box spring or frame of the bed. The mattress (5) is placed over the pad (1). The cables (4) lead out from under the mattress (5) and up to the processing and control unit (2). The patient, when placed on the mattress (5), can be monitored for a heartbeat and respiration.

The present invention works as follows:

The pad (1), with its array of sensors (3) is placed under the mattress (5), all of the cables (4) are connected between the sensors (3) and the processing and control unit (2), and the system is turned on. The system requires at least two sensors, however, if it is anticipated that the patient will move about, such as an infant in a crib, at least three sensors should be used.

Upon initial use, the system must be calibrated and the sensitivity of each sensor must be adjusted to filter out ambient noise, floor vibrations, and other environmental activities. Once the patient is placed on the bed an Fast Fourier Transform (FFT) algorithm will be used in order to adjust the filters to the individual heart rate and respiration rate of the patient, using an axial signal (the difference between the signals received from the upper and lower body, described in further, detail below) as an input. This procedure will identify and determine the unique heart and respiration rates of the patient and as a result will make modifications to the frequency bands that are being filtered in order to detect individual heartbeats and respirations. Additional calibrations are then made (e.g. using the average amplitudes of the vital sign signals in order to set the sensitivity threshold for the detecting algorithms). The system must be recalibrated for each patient to accommodate the unique vital signs and other movements of each patient, as well as any changes in ambient noise.

When the patient is on the bed, he may be lying so that the body's center of gravity is not located at the center of the mattress, which is also the center of the housing of the present invention. Where this is the case, the present invention normalizes the center of gravity of the patient to the center of the mattress using environmental noise such as vibrations signals from the floor, thus eliminating the effect of the patient's location between the sensors from the measurements. The added signal manifests in a different manner at each sensor when the subject is located at a place other than the center of the mattress. By analyzing the difference in amplitudes of the signal at each sensor, the real center of gravity is detected, and in dividing the wanted signal (pressure) collected at each sensor by the amplitude of the environmental component of the collected signal, the center of gravity is virtually moved to the center of the mattress.

Once the calibrations are made, and the patient is on the bed, the sensors (3) are able to collect data for transmission to the processing and control unit (2). Each sensor (3) gathers signals generated by the vertical and horizontal movements of the patient's mass, and by the vertical environmental noises.

The sum of all of the signals from each sensor (3) or group of sensors (3) provides a combined signal corresponding to the vertical movements of the body, mainly respiratory movements, and to the vertical environmental or ambient noise, mainly the product of a vibrating floor. This is referred to as a “Vertical” signal.

Subtracting the signals collected by each sensor (3) or group of sensors (3) from each other results in a combined signal corresponding to horizontal movements of the body's center of gravity, mainly due to blood circulation and diaphragm movements caused by respiration. This difference of signals is referred to as the “Horizontal” signal.

In order to determine the heart rate, the signals to be subtracted from each other are those corresponding to the signals received from the sensor located under the upper body and the signals received from the sensor located under the lower body of the patient. This is because the blood's center of gravity, driven by the heart, moves along the body's axis. The “Axial” signal describes this difference between the signals received from the upper body and the lower body, which is the Horizontal signal when measured along the body axis. Determining the Axial signal is a crucial step in detecting and determining heart rate.

When the major position of the patient is stationary, as with adults in a hospital bed, or a baby in a small crib, the best solution will include two sensors (3), or two groups of sensors (3), whose connecting line is parallel to the body's axis. Under these conditions, the Axial signal will be the absolute value of the difference between the signals of the two sensors (3), or between the combined signals of two pairs or two groups of sensors (3).

When the patient is expected to change the direction of the body axis, like a baby in a bed or a cradle, the best solution will consist of an array of at least three sensors (3). In this case, since the body axis is not known, the Axial signal is determined by mathematically determining the maximum difference of the signals within each pair of sensors (3). This maximal difference is referred to here as the Axial signal.

The processing and control unit (2) processes all of the input signals and computes the Axial and Vertical signals in accordance with the methods described above. Then, using analog or digital filters to isolate heart rate and respiration artifacts from each other and to filter out background noises, the cyclical vital signs are measured and optionally displayed. Another algorithm, whose inputs are the heart rate and respiration rate, is used to trigger an alarm system in the event that the heart rates or respiration rates or both fall within a predefined range, said system being an integral part of the processing and control unit (2).

The best solution is achieved by filtering, normalizing, and comparing the Vertical and Axial signals. This enables accurate heart rate and respiration rate detection and determination, while eliminating limb movements and other external artifacts.

Filtering signals in order to detect and determine heart rate should be done using high-pass filters, (to filter out low frequencies), whose frequency is at least twice the monitored patient's typical heart frequency, contrary to previous, unsuccessful efforts known to detect and determine heart rate using high-pass filter whose cut off frequency, or threshold, is lower than the heart rate. This makes use of the fact that the typical contraction time of the heart, causing the mechanical signal, is less than a fifth of the cycle time (time divided by the heart rate). Using a low-pass filter (to filter out high frequencies) whose frequency is at least 6 times the typical heart rate helps to reduce noise.

The peaks of the Axial signal can be used to first detect the patient's heartbeat and then determine the rate.

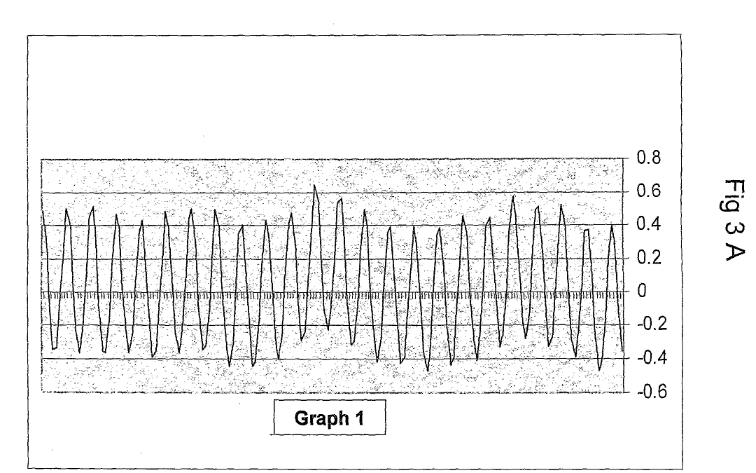

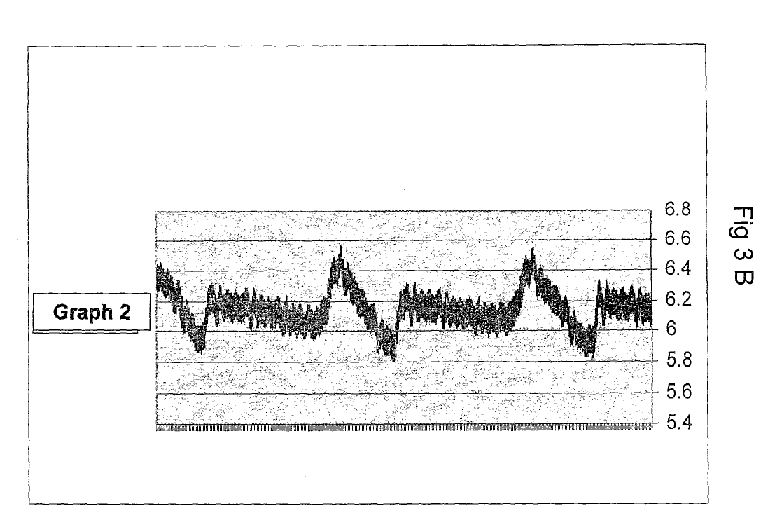

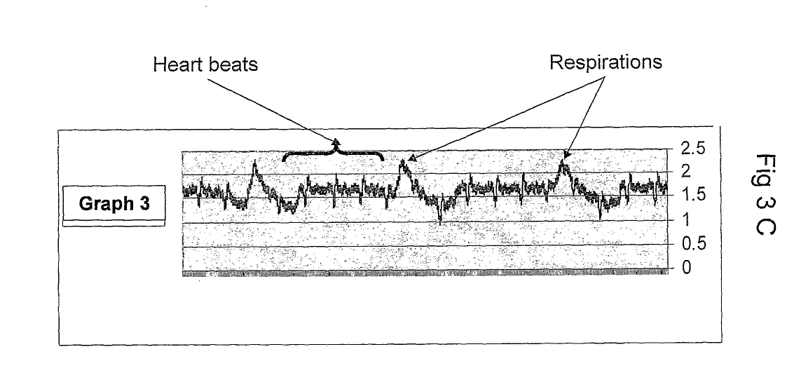

FIGS. 3A, 3B, and 3C demonstrate the results of this process, by way of three graphs, where FIGS. 3A and 3B show signals collected by prior art systems, and FIG. 3C shows data collected by the present invention. FIG. 3A shows a half-second signal collected by a prior art monitor using a single sensor. This graph shows that the heartbeat is masked by the dominant environmental background noise. FIG. 3B shows a 10-second signal collected by a prior art monitor, where respiration, represented by the large peaks, begins to reveal itself. However, the heartbeats remain invisible due to clutter from the environmental signals. For both prior art examples, the same results are achieved when using either a single sensor or the sum from the signals of multiple sensors. FIG. 3C shows that, in the same 10-second period, by subtracting signals collected from two sensors, the present invention is able to suppress the environmental signals and clearly detect and identify both respiration rate, represented by the large peaks, and heartbeats, represented by the small peaks.

The present invention includes a presence detection function in the processing unit using the same or other sensors, that shows whether the patient is on the bed or not, optionally triggering a “missing patient” alarm, and that prevents triggering a false “no heart beat/no respiration” alarm.

In an alternative embodiment, the sensors (3) can have opposite, or different, polarizations so that combining the output signals of each sensor or array of sensors provides the difference required to determine the heart and respiration rates.

While the above description contains many specificities, these should not be construed as limitations on the scope of the invention, but rather as exemplifications of the preferred embodiments. Those skilled in the art will envision other possible variations that are within the scope of the invention. Accordingly, the scope of the invention should be determined not by the embodiment illustrated, but by the appended claims and their legal equivalents.

What is claimed is:

1. A method for non-invasive monitoring of subject heartbeat rate, said method comprised of:
Collecting pressure changes received from at least two sensors located beneath the subject's body;
Finding the difference between at least two sensor signal measurements;
analyzing the difference signal for identifying and detecting heartbeats or heart rate.

2. The method of claim 1 further comprising the step of filtering the calculated difference signal for reducing background noise and respiratory artifact and other body movements in accordance with predefined signal frequency band values.

3. The method of claim 1 further comprising the step of identifying the respiration rate.

4. The method of claim 1 further comprising the step of calculating the sum of at least two signal measurements and filtering and analyzing the calculated sum signal in combination with the difference signal for identifying and detecting the heartbeat rate and respiration rate.

5. The method of claim 1 further comprising the step of calculating the maximum difference signal between sets of sensors, wherein the identification and detection of the heartbeat rate is based on said maximum signal difference.

6. The method of claim 1 further comprising the step of calibration for calculating the pre-defined filter signal frequency band values, wherein calibration is based on the FFT algorithm.

7. The method of claim 1 wherein the filtering is preformed by a high pass filter, wherein the cut off frequency is twice a pre-defined heart rate.

8. The method of claim 1 wherein the analyzing includes identifying peak values of the filtered signal.

9. The method of claim 1 wherein at least one sensor is located beneath the lower part of the subject's body and at least one sensor is located beneath the upper part of the subject's body.

10. The method of claim 1 wherein the difference signal represents the horizontal movements of the subject and the analyzing includes detection of blood circulation.

11. A system for non-invasive monitoring of subject heartbeat rate, said system comprised of:
at least two sensors located beneath the subject's body for measuring pressure changes;
an electronic mechanism for finding the difference between at least two sensor signal measurements;
a processing module for analyzing the difference signal to identify and detect the heartbeats or heart rate.

12. The system of claim 11 further comprising a filtering module for reducing background noise of the difference signal in accordance with pre-defined signal frequency band values

13. The system of claim 11 wherein the processing module further identifies the respiration rate

14. The system of claim 11 wherein the electronic mechanism further calculates the sum of at least two signal measurements and the processing module further analyzes the calculated sum signal in combination with the difference signal for identifying and detecting the heartbeat rate and respiration rate.

15. The system of claim 11 wherein the electronic mechanism further calculates the maximum difference signal between sets of sensors, wherein the identification and detection of the heartbeat rate is based on said maximum signal difference.

16. The system of claim 11 further comprising a calibration module for calculating the pre-defined signal frequency band values, wherein calibration is based on the FFT algorithm.

17. The system of claim 11 wherein the filtering module is a high pass filter, wherein the cut off frequency is twice a pre-defined heart rate.

18. The system of claim 11 wherein at least one sensor is located beneath the lower part of the subject's body and at least one sensor is located beneath the upper part of the subject's body.

19. The system of claim 11 wherein the analyzing includes identifying peak values of the filtered signal.

20. The system of claim 11 wherein the difference signal represents the horizontal movements of the subject and the filtering and analyzing includes detection of the blood circulation.

21. The system of claim 11 wherein the sensors are integrated within a single rigid housing.
